(19) United States
(12) Patent Application Publication
(10) Pub. No.: US 2025/0114588 A1
(43) Pub. Date: Apr. 10, 2025

(54) Port to Catheter Connection System

(71) Applicant: Bard Peripheral Vascular, Inc., Franklin Lakes, NJ (US)

(72) Inventors: Jessica Hoye, Phoenix, AZ (US); Bryon Ray Densley, Rochester, MN (US); Christian Andersen, Queen Creek, AZ (US); Ian N. Thomas, West Bountiful, UT (US); Diana Fiumefreddo, Salt Lake City, UT (US)

(21) Appl. No.: 18/290,751

(22) PCT Filed: Jul. 27, 2021
(86) PCT No.: PCT/US21/43310

§ 371 Date: Jan. 19, 2024

(51) Int. Cl. A61M 39/12

(52) U.S. Cl. CPC A61M 39/12

(57) ABSTRACT

Embodiments disclosed herein are directed to a cathlock system integrally formed with a port or similar access device. The cathlock mechanism can include a shroud defining a recess, a stem disposed therein, and a collet lock transitionable between an unlocked and a locked configuration. The system can further include a tool configured to engage an outer surface of the cathlock and plastically deform the collet lock into the recess to the locked configuration. The collet lock can compress the catheter onto the stem to form a fluid-tight seal therebetween. The collet lock can be configured to engage the cathlock mechanism in the open configuration to facilitate coupling the catheter with the cathlock within the confined environment of a subcutaneous placement. Embodiments can include an extension leg disposed between the port and the cathlock mechanism.

### SUMMARY

Briefly summarized, embodiments disclosed herein are directed to a port and catheter connection system, and associated methods thereof. The connection system can include an integrated, crimpable, cathlock mechanism configured to couple a catheter to a stem of a port, and a tool configured to transition the cathlock mechanism between the unlocked configuration and the locked configuration.

Proximally trimmable catheters allow for post-placement sizing of the catheter. When placing a catheter and port assembly, the position of the distal tip of the catheter can be important for the efficacy of the treatment. For example, when placing a catheter within the superior vena cava, if the distal tip of the catheter falls short of the target area, the efficacy of the medicament is reduced. If the distal tip is advanced too far, the distal tip can cause arrhythmia. The distance between the distal tip of the catheter and the port can vary since the distances between the target location, insertion site to the vasculature, and the location of the port can vary between patients and procedures. Estimating the catheter length before placement can lead to errors that result in misplacement of the distal tip.

Proximally trimmable catheters allow for placement of the catheter distal tip at the target location before trimming a proximal portion of the catheter to the correct length. The clinician can then attach the catheter to a subcutaneous port, or similar access device. However, securing the catheter to the port can be challenging. The connection must be leak-proof, especially under high-pressure infusion. Further, manipulating the catheter and port within the confined, wetted environment of a subcutaneous access site can lead to slippage, undue trauma to the access site, or misplacement of the catheter distal tip.

Disclosed herein is a system for coupling a catheter to a port including, a catheter defining a first diameter, a port including a shroud extending axially from the port and defining a recess having a second diameter larger than the first diameter, the recess including a stem extending axially and configured to engage a lumen of the catheter, a collet lock extending annularly about the stem axis and configured to transition between an unlocked configuration and a locked configuration when an axial force is applied, and a tool configured to engage the shroud and the collet lock and apply an axial force to transition the collet lock from the unlocked configuration to the locked configuration.

In some embodiments, a portion of the collet lock is retained within the recess, in the unlocked configuration, an outer surface of the collet lock engages an inner surface of the recess in one of an interference fit, press-fit, or snap-fit engagement. In some embodiments, the collet lock includes a plurality of fingers configured to plastically deform radially inward when transitioned to the locked configuration. In some embodiments, the plurality of fingers impinge on a surface of the recess when the axial force is applied to transition the collet lock from the unlocked configuration to the locked configuration. In some embodiments, a tip of a finger of the plurality of fingers engages an outer surface of the catheter in the locked configuration, compressing the catheter onto the stem.

In some embodiments, a tip of the plurality of fingers define a diameter that is larger than the first diameter of the catheter. In some embodiments, the tool includes a first arm hingedly coupled to a second arm, a tip of the first arm includes a first fork configured to engage the port, and a tip of the second arm includes a second fork configured to engage the collet lock. In some embodiments, one or both of the first fork and the second fork includes a first tine and a second tine defining a notch having a first width, the first width being equal to or larger than the first diameter of the catheter. In some embodiments, the shroud includes a channel defining a third diameter being equal to or less than the first width, the first fork engaging the channel of the shroud in one of an interference fit, press-fit, or snap-fit engagement.

In some embodiments, one or both of the first fork and the second fork includes an indentation extending along an edge of the notch and defining a second width, the second with being larger than an outer diameter of the collet lock, a surface of the indentation engaging the collet lock with the axial force is applied. In some embodiments, the system further includes an extension leg extending between the shroud and the port, formed integrally therewith, and providing fluid communication therebetween, the extension leg formed of a flexible material. In some embodiments, the port includes a reservoir in fluid communication with the stem and having a needle penetrable septum thereover.

Also disclosed is a method of coupling a catheter to a port including, urging the catheter over a stem of the port, engaging a first engagement tip of a tool with a shroud of the port, engaging a second engagement tip of the tool with a collet lock, urging the first engagement tip towards the second engagement tip along an axis of the stem, and plastically deforming the collect lock from an open configuration to a closed configuration to secure the catheter to the stem.

In some embodiments, an outer surface of the collet lock in the open configuration engages an inner surface of a recess in one of an interference fit, press-fit, or snap-fit engagement, the recess defined by the shroud of the port. In some embodiments, the method further includes plastically deforming a plurality of fingers of the collet lock radially inward to transition the collect lock from the open position to the closed position, the plurality of fingers impinging on a surface of the recess. In some embodiments, the method further includes compressing the catheter onto the stem by engaging a tip of a finger of the plurality of fingers with an outer surface of the catheter. In some embodiments, a tip of the plurality of fingers define a diameter that is larger than a diameter of the catheter.

In some embodiments, one or both of the first engagement tip and the second engagement tip includes a first tine and a second tine defining a notch having a first width, the first width being larger than the diameter of the catheter. In some embodiments, the shroud includes a channel defining a channel diameter being equal to or less than the first width, the first engagement tip engaging the channel of the shroud.

In some embodiments, one or both of the first engagement tip and the second engagement tip includes an indentation extending along an edge of the notch and defining a second width, the second width being larger than an outer diameter of the collet lock, a surface of the indentation engaging the collet lock when the first engagement tip is urged towards the second engagement tip. In some embodiments, the method further includes an extension leg extending between the shroud and the port, formed integrally therewith, and providing fluid communication therebetween, the extension leg formed of a flexible material. In some embodiments, the port includes a reservoir in fluid communication with the stem and having a needle penetrable septum thereover.

Also disclosed is a catheter and port coupling system including, a catheter defining a first diameter, a port including a shroud extending axially from the port and defining a recess having a second diameter larger than the first diameter, the recess including a stem extending axially and configured to engage a lumen of the catheter, and a collet lock extending annularly about the stem axis and configured to transition between an unlocked configuration and a locked configuration when an axial force is applied to secure the catheter to the port.

In some embodiments, the system further includes an extension leg extending between the shroud and the port, formed integrally therewith, and providing fluid communication therebetween, the extension leg formed of a flexible material. In some embodiments, a portion of the collet lock is retained within the recess in the unlocked configuration, an outer surface of the collet lock engages an inner surface of the recess in one of an interference fit, press-fit, or snap-fit engagement. In some embodiments, the collet lock includes a plurality of fingers configured to plastically deform radially inward when transitioned to the locked configuration.

In some embodiments, the plurality of fingers impinge on a surface of the recess when the axial force is applied to transition the collet lock from the unlocked configuration to the locked configuration. In some embodiments, a tip of a finger of the plurality of fingers engages an outer surface of the catheter in the locked configuration, compressing the catheter onto the stem. In some embodiments, a tip of the plurality of fingers define a diameter that is larger than the first diameter of the catheter. In some embodiments, the port includes a reservoir in fluid communication with the stem and having a needle penetrable septum thereover.

In some embodiments, the system further includes a tool configured to engage the shroud and the collet lock and apply an axial force to transition the collet lock from the unlocked configuration to the locked configuration. In some embodiments, the tool includes a first arm hingedly coupled to a second arm, a tip of the first arm includes a first fork configured to engage the port, and a tip of the second arm includes a second fork configured to engage the collet lock. In some embodiments, one or both of the first fork and the second fork includes a first tine and a second tine defining a notch having a first width, the first width being equal to or larger than the first diameter of the catheter. In some embodiments, the shroud includes a channel defining a third diameter being equal to or less than the first width, the first fork engaging the channel of the shroud in one of an interference fit, press-fit, or snap-fit engagement. In some embodiments, one or both of the first fork and the second fork includes an indentation extending along an edge of the notch and defining a second width, the second with being larger than an outer diameter of the collet lock, a surface of the indentation engaging the collet lock with the axial force is applied.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which.

FIGS. 1A-1B show perspective cross-sectional views of a port including an integrated, crimpable, cathlock mechanism, in accordance with embodiments disclosed herein.

FIG. 2A shows a cross-section side view of the cathlock mechanism of FIG. 1 in an unlocked position, in accordance with embodiments disclosed herein.

FIG. 2B shows a cross-section side view of a cathlock mechanism in an unlocked position including a tool engaged therewith, in accordance with embodiments disclosed herein.

FIG. 2C shows a cross-section plan view of a cathlock mechanism in an unlocked position including a tool engaged therewith, in accordance with embodiments disclosed herein

FIG. 2D shows a cross-section side view of a cathlock mechanism in a locked position including a tool engaged therewith, in accordance with embodiments disclosed herein.

FIG. 2E shows close up detail of the cathlock mechanism of FIG. 2A, in accordance with embodiments disclosed herein.

FIG. 3A shows an axial view of a collet lock of the cathlock mechanism of FIG. 1, in accordance with embodiments disclosed herein.

FIG. 3B shows a side view of a collet lock of the cathlock mechanism of FIG. 1, in accordance with embodiments disclosed herein.

FIG. 4A shows a perspective view of a tool configured to transition a cathlock mechanism between a locked and an unlocked position, in accordance with embodiments disclosed herein

FIG. 4B shows close up detail of a side view of the tool of FIG. 4A, in accordance with embodiments disclosed herein.

FIG. 4C shows close up detail of an end view of the tool of FIG. 4A, from the perspective of a second end of the tool, in accordance with embodiments disclosed herein.

FIG. 4D shows close up detail of an axial view of the tool of FIG. 4A, in accordance with embodiments disclosed herein.

FIG. 5 shows an axial view of an inside surface of a first arm of the tool of FIG. 4A including the collet lock engaged therewith, in accordance with embodiments disclosed herein

FIG. 6 shows a cross-section side view of a cathlock mechanism including an extension leg, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

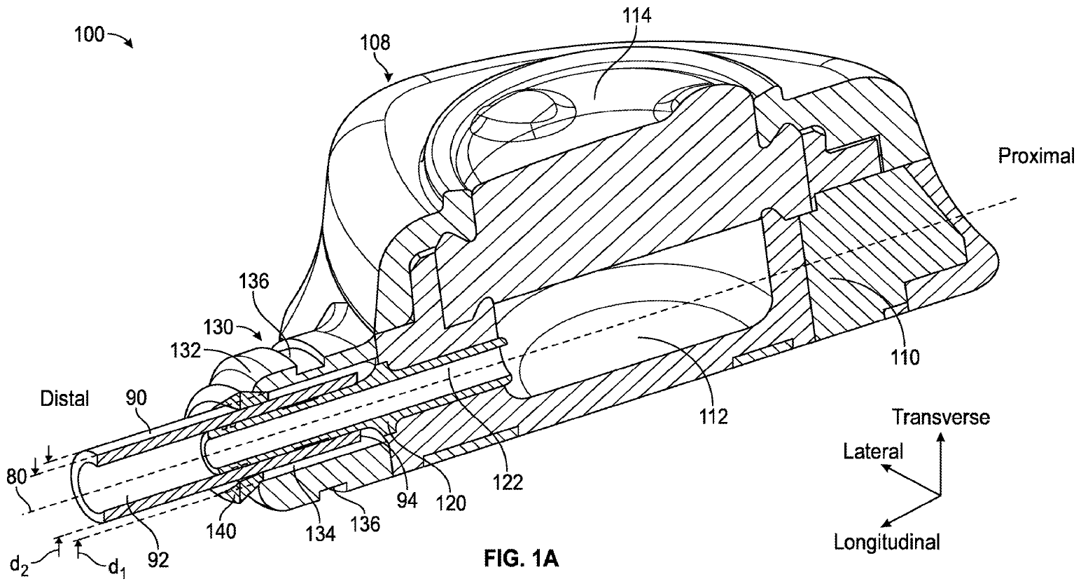

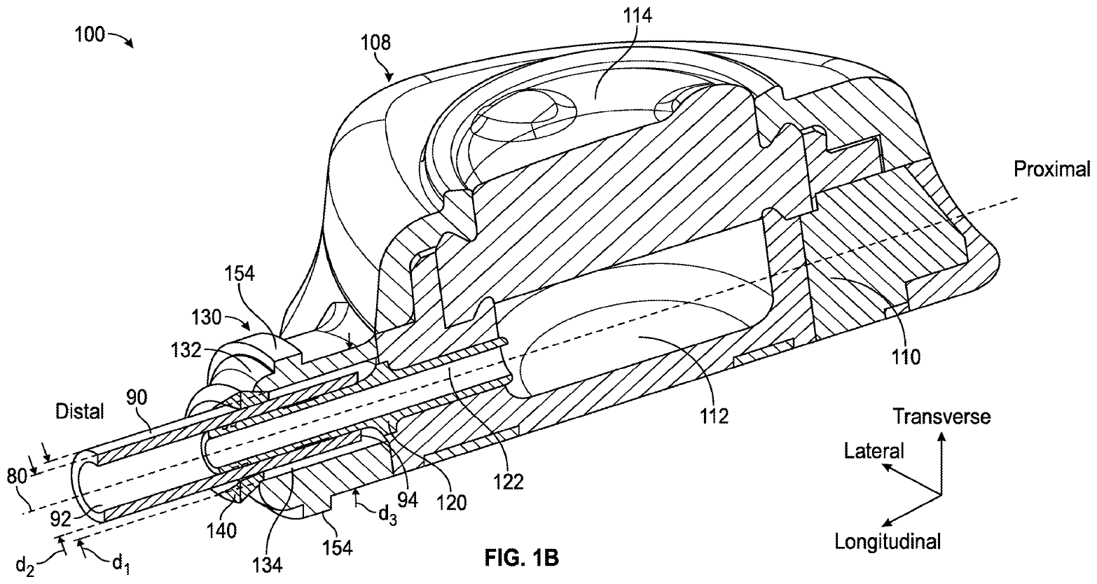

To assist in the description of embodiments described herein, as shown in FIG. 1, a longitudinal axis extends substantially parallel to an axial length of the stem 120. A lateral axis extends normal to the longitudinal axis, and a transverse axis extends normal to both the longitudinal and lateral axes. As used herein, a horizontal plane extends along the lateral and longitudinal axes. A vertical plane extends normal to the horizontal plane.

Unless defined otherwise, all technical and scientific terms used herein have the same meaning as commonly understood by those of ordinary skill in the art.

FIGS. 1A-1B show embodiments of a port connector system 100 including a port 108 having a crimpable, catheter locking (“cathlock”) mechanism 130 configured to secure a catheter 90 to a stem 120. FIGS. 1A-1B show perspective cross-section views of the port 108, cathlock 130, and catheter 90. The port 108 can generally include a port body 110 that defines a reservoir 112 and can include a needle penetrable septum 114 disposed thereover. The septum 114 can be configured to provide percutaneous access to the reservoir 112 by an access needle. The access needle can penetrate the skin surface and underlying tissues and can be urged through the needle penetrable septum 114 to access the reservoir 112 and provide fluid communication therewith.

The port 108 can further include a stem 120 defining a stem lumen 122 that is in fluid communication with the reservoir 112. The stem 120 can define a central axis 80 that extends parallel to the longitudinal axis. In an embodiment, a proximal end 94 of the catheter 90 can be urged over the stem 120 to provide fluid communication between the reservoir 112 and a lumen 92 of the catheter 90. A distal tip of the catheter 90 can be disposed within a vasculature of a patient to provide fluid communication therewith. It will be appreciated that the subcutaneous port 108 is an exemplary access device and that embodiments disclosed herein can be used with various ports or similar access devices.

In an embodiment, the port 108 can include a crimpable, cathlock mechanism 130 integrally coupled therewith. The cathlock mechanism 130 can include a shroud 132, extending distally from the port body 110 and extending annularly about the stem 120. In an embodiment, a distal tip of the shroud 132 can extend to a point that is proximal of the distal tip of the stem 120. In an embodiment, a distal tip of the shroud 132 can extend to a point that is distal of the distal tip of the stem 120. In an embodiment, a distal tip of the shroud 132 and a distal tip of the stem 120 can extend equidistant from the body 110.

In an embodiment, the shroud 132 can define a substantially cylindrical recess 134 extending longitudinally from the distal tip of the shroud 132 and can include the stem 120 disposed therein. In an embodiment, the recess 134 can define a substantially circular or elliptical cross-sectional shape. However, other cross-sectional shapes are also contemplated. In an embodiment, the recess 134 can define a first diameter (d1) which can be larger than an outer (second) diameter (d2) of the catheter 90. In an embodiment, as shown in FIG. 1A, the shroud 132 can include a channel 136 disposed on an outer surface thereof and extending annularly about a portion of the shroud 132, about the central axis 80. The channel 136 can define a third diameter (d3). The channel 136 can be configured to receive a portion of a tool 160 therein, as described in more detail herein. In an embodiment, as shown in FIG. 1B, the outer surface of the shroud 136 can define a third diameter (d3) and the shroud 132 can include a flange 154 extending radially therefrom, relative to the central axis 80. A portion of the tool 160 can engage the outer surface of the shroud 132 between the flange and the port body 110. The tool 160 can then abut against the flange 154, as described in more detail herein.

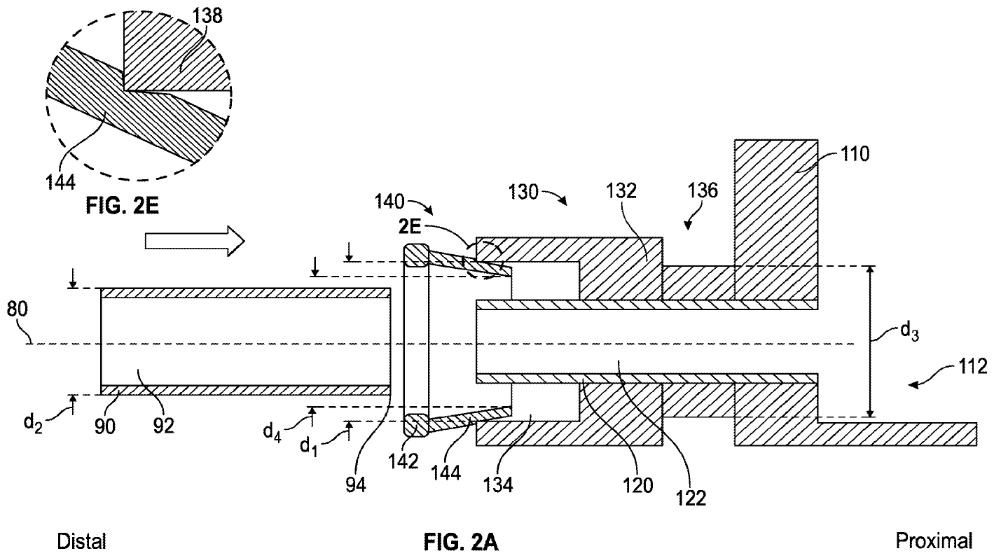

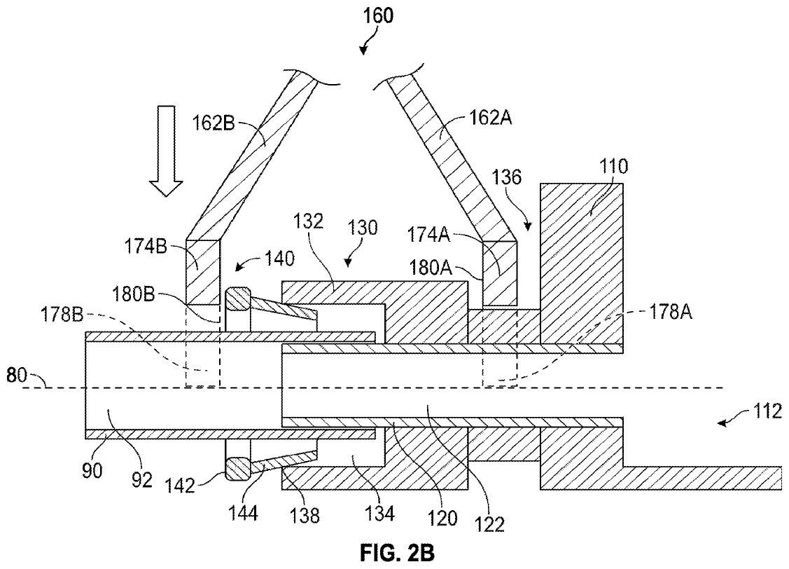

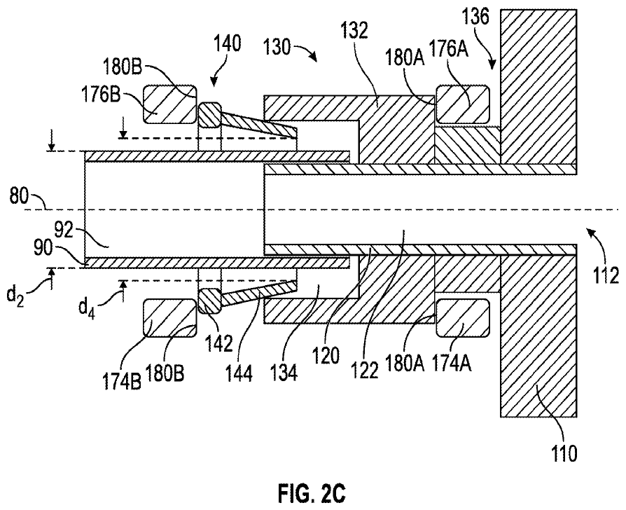

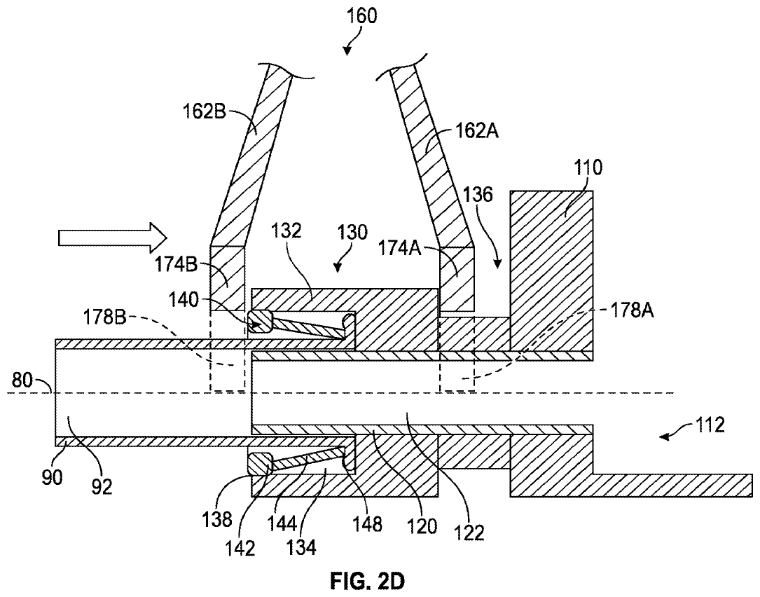

In an embodiment, the cathlock mechanism 130 can further include a crimpable, collet lock 140 configured to be plastically deformed, or “swaged,” from an unlocked, or open, configuration to a locked, or closed, configuration to engage an outer surface of the catheter 90 and secure the catheter 90 to the stem 120. In an embodiment, the collet lock 140 can slidably engage an outer surface of the catheter 90 and can be coupled with the recess 134 in a locked configuration, in an interference fit, press-fit, or snap fit engagement. In an embodiment, as shown in FIG. 2A, the collet lock 140 in the unlocked configuration can engage an entrance 138 of the recess 134 in an interference fit, press-fit, or snap-fit engagement, and can be retained therein. As such, the collet lock 140 can be retained by the cathlock mechanism 130 prior to the catheter 90 engaging the cathlock mechanism 130. Advantageously, the cathlock mechanism 130, with the collet lock 140 retained therein, can form a single functional unit without the user having to assemble different portions of the cathlock mechanism 130 prior to engaging the catheter 90 therewith. This can be of particular importance within the confined, and naturally lubricated environment of a subcutaneous placement.

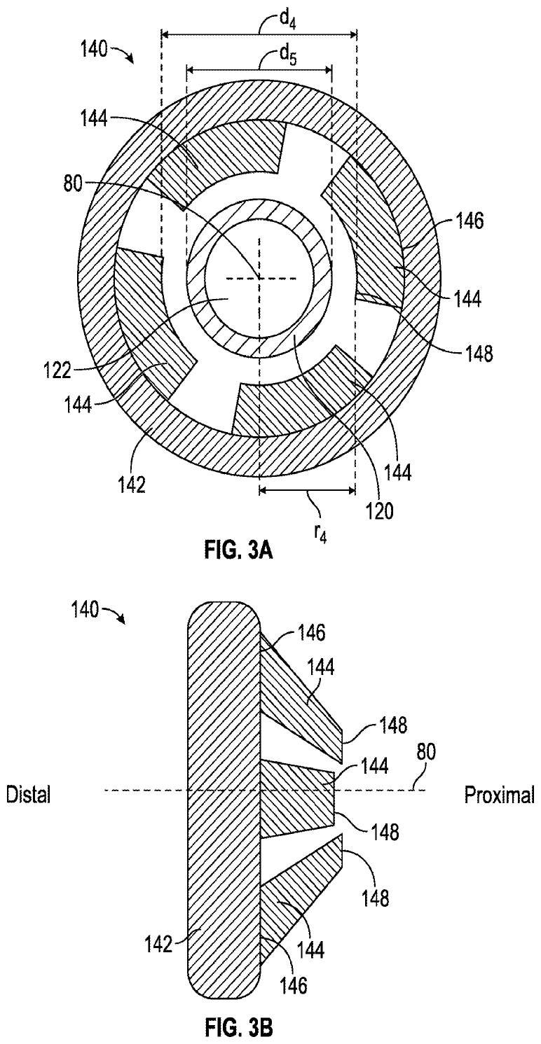

FIGS. 3A-3B show further details of the collet lock 140. FIG. 3A shows an axial view of the collet lock 140, FIG. 3B shows a side view. In an embodiment, the collet lock 140 can include a ring 142 extending annularly about the central axis 80 of the stem 120. In an embodiment, an outer diameter of the ring 142 can be equal to, larger than, or less than the inner diameter (d1) of the recess 134. The collet lock 140 can further include and one or more fingers 144 extending radially inward from a surface of the ring 132, e.g. an inner surface or a side surface of the ring 142. Each finger of the plurality of fingers 144 can be coupled to the ring 142 at abase 146 and extend radially inward to a tip 148. In an embodiment, the one or more fingers 144 can extend at an angle, relative to the central axis 80. In an embodiment, the one or more fingers 144 can extend at an angle extending towards the port body 110, i.e. in a proximal direction. In an embodiment, the collet lock 140, or a portion thereof, can be formed of a malleable, or plastically deformable, material such as plastic, polymer, metal, alloy, composite, or the like.

In an embodiment, as shown in FIG. 3A, the tips 148 of the one or more fingers 144 can co-operate to define a diameter (d4). Worded differently, a radius (r4) extending from the central axis 80 to the tip 148 can be half that of the diameter (d4). The diameter (d4) can be less than the diameter (d1) of the recess 134. In an embodiment, the diameter (d4) can be larger than outer diameter (d2) of the catheter 90. As such, as shown in FIG. 2A, the catheter 90 can pass through the collet lock 140 and engage the stem 120 without the catheter 90 engaging the collet lock 140 or without the collet lock 140 impeding axial movement of the catheter 90.

In an embodiment, the diameter (d4) can be equal to or less than the outer diameter (d2) of the catheter 90. In an embodiment, the one or more fingers 144 can be configured to elastically deform radially outward, to receive the catheter 90 therebetween. In an embodiment, the tip 148 of the one or more fingers 144 can elastically deform radially outward to engage an outer surface of the catheter 90 in an interference fit. As such, the plurality of fingers 140 can engage the catheter 90 in an interference fit. Advantageously, the collet lock 140, retained within the cathlock mechanism 130, can slidably engage the catheter 90 in the unlocked or open position and retain the catheter 90 relative to the cathlock mechanism 130 prior to transitioning the collet lock 140 to the locked position.

In an embodiment, the diameter (d4) can be equal to or less than an outer diameter (d5) of the stem 120. As such, the tip 148 of the one or more fingers 144 can elastically deform radially outward to engage an outer surface of the stem 120 in an interference fit. Advantageously, the collet lock 140 can engage the stem 120 to retain the collet lock 140 within the cathlock mechanism 130, in the unlocked configuration. In an embodiment, the catheter 90 can be urged into the recess 134, between the plurality of fingers 144 and the outer surface of the stem 120, elastically deforming the plurality of fingers 144 radially outward to allow the catheter 90 to engage the stem 120. The cathlock mechanism 130 can then be transitioned to the locked configuration, as described herein.

In an embodiment, as shown in FIG. 2E, an outer surface of the one or more fingers 144 can engage a surface of the recess 134, e.g. an entrance 138, in an interference fit, press-fit, or snap fit engagement, in the unlocked position. In an embodiment, the one or more fingers 144 can elastically deform radially inward, an outer surface of the one or more fingers 144 and can include a protrusion, detent, clip, barb, or similar structure configured to engage the recess 134 in a snap-fit engagement. Advantageously, recess 134 can retain the collet lock 140 within the recess in the unlocked position.

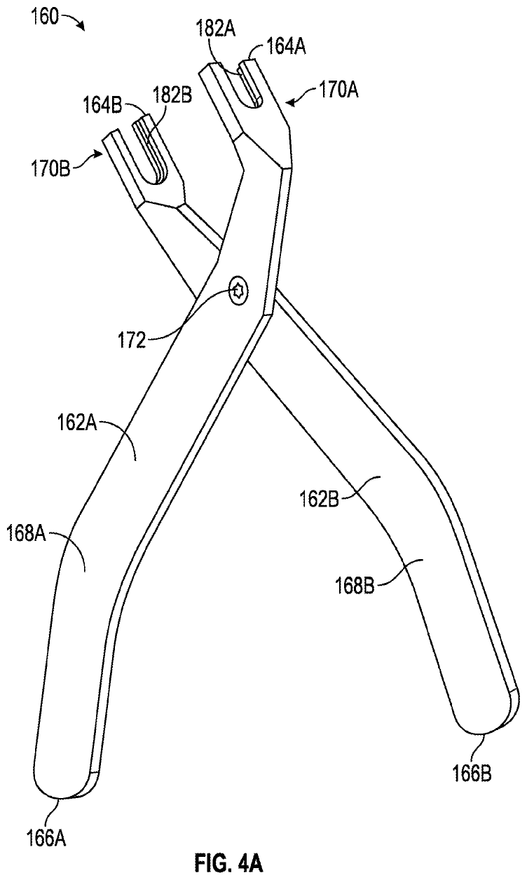

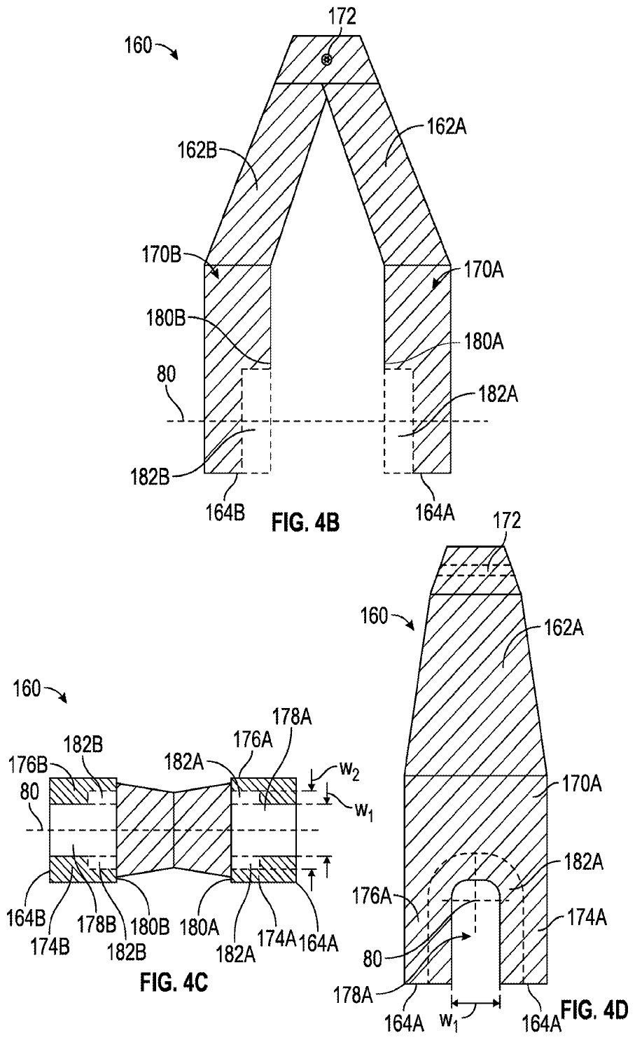

In an embodiment, as shown in FIGS. 4A-4D, the port connector system 100 can further include a tool 160 configured to swage the collet lock 140 into the recess 134 to transition the cathlock mechanism 130 from the unlocked configuration to the locked configuration and secure the catheter 90 to the stem 120. In an embodiment, the tool 160 or portions thereof, can be formed of a plastic, polymer, metal, alloy, composite, combinations thereof, or the like. To note, FIG. 4A shows a perspective view of the tool 160, FIG. 4C shows an end view of the tool 160 as viewed from the second end 164. FIGS. 4B and 4D show close up detail of a portion of the tool 160, extending between the hinge 172 and the second end 164.

In an embodiment, the tool 160 can include a first arm 162A and a second arm 162B hingedly coupled to each other. Each arm 162 can extend between a first end 164 and a second end 166, i.e. a first arm 162A can extend from a first arm first end 164A to a first arm second end 166A, and a second arm 162B can extend from a second arm first end 164B to a second arm second end 166B. Each arm 162 can include a handle portion 168 disposed proximate the second end 166 and an engagement tip 170, or “fork,” disposed proximate the first end 164. The first arm 162A can be hingedly coupled to the second arm 162B at a hinge 172 disposed at a point between the first end 164 and the second end 166. As such, rotating the first handle 168A towards the second handle 168B can cause the first engagement tip 170A to rotate towards the second engagement tip 170B.

In an embodiment a distance between the hinge 172 and the second end 166 can be larger than a distance between the hinge 172 and the first end 164. As such, the tool 160 can provide mechanical advantage to the compression between the first engagement tip, or “first fork,” 170A and the second engagement tip, or “second fork” 170B when the first handle 168A is rotated towards the second handle 168B.

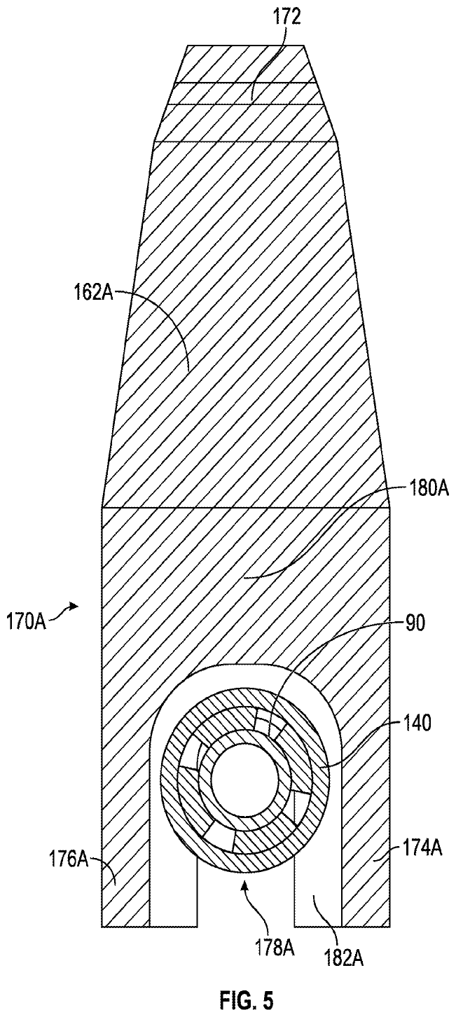

As shown in FIGS. 4B-4D, each engagement tip, or fork, 170 can include a first tine 174 and a second tine 176 extending away from hinge 172 and defining a notch 178 therebetween. In an embodiment, a width (w1) of the notch 178 can be equal to, larger than, a diameter (d3) of the channel 136 or shroud 132. As such, the notch 178 can engage the shroud 132 in an interference fit or press-fit engagement. In an embodiment, a width (w1) of the notch 178 can be equal to larger than a diameter (d2) of the catheter 90. In an embodiment, as shown in FIGS. 4B-5, an inner surface 180 of the engagement tip 170 can include an indentation 182 extending along an edge of the notch 178. The indentation 182 can define a second width (w2) that is larger than the first width (wil). The second width (w2) can be equal to or larger than an outer diameter of the ring 142 of the collet lock 140 (FIG. 5). In an embodiment, the indentation 182 can be configured to receive the ring 142 therein and mitigate lateral or transverse movement of the collet lock 140 relative to the tool 160. In an embodiment, the indentation 182 can be configured to engage the ring 142 in an interference fit, press-fit, or snap fit engagement. Advantageously, the indentation 182 can mitigate accidental slippage of the collect lock 140 relative to the tool 160 when an axial force is applied.

In an exemplary method of use, the port system 100 is provided including the crimpable, collet lock 140 and a tool 160, as described herein. As shown in FIG. 2A, a catheter 90 can be inserted into the cathlock mechanism 130, through the collet lock 140, into the recess 134 and onto the stem 120 to engage the stem 120 in an interference fit. In an embodiment, the collet lock 140 can be retained within the recess 134 by an outer surface of the collet lock 140 engaging a surface of the recess 134, e.g. entrance 138. In an embodiment, the plurality of fingers 144 of the collect lock 140 can engage the catheter 90 in an interference fit in the unlocked configuration and can mitigate retrograde movement of the catheter 90 from the recess 134. In an embodiment, the plurality of fingers 144 can engage the stem 120 in an interference fit to retain the collet lock 140 within the recess 134.

As shown in FIGS. 2B-2C, the tool 160 can engage the cathlock mechanism 130. FIG. 2B shows a longitudinal vertical cross-sectional view, extending along the central axis 80 of the stem 120. FIG. 2C shows a longitudinal horizontal cross-sectional view, extending along the central axis 80 of the stem 120. In an embodiment, a first engagement tip 170A of the first arm 162A can engage the channel 136 and/or shroud 132 and the second engagement tip 170B of the second arm 162B can engage the collet lock 140 and/or catheter 90. In an embodiment, a first engagement tip 170A of the first arm 162A can engage the collet lock 140 and/or catheter 90 and the second engagement tip 170B of the second arm 162B can engage the channel 136 and/or shroud 132.

In an embodiment, as shown in FIG. 2B, the channel 136, or a portion of the shroud 132, can be received within the first notch 178A and a first inner surface 180A of the first engagement tip 170A can engage a surface of the shroud 132 or the flange 154. Further, a portion of the catheter 90 can be received within the second notch 178B and a second inner surface 180B of the second engagement tip 170B can engage a surface of the collet lock 140, e.g. ring 142. In an embodiment, the notch 178 can engage one of the catheter 90, channel 136 or shroud 132 in an interference fit, or press-fit engagement. In an embodiment, the notch 178 can include a protrusion (not shown) extending into the notch 178 from a surface thereof. As such, the notch 178 can engage one of the catheter 90, channel 136 or shroud 132 in a snap-fit engagement.

As shown in FIG. 2D, the tool 160 can apply an axial force to the cathlock mechanism 130 by urging the first engagement tip 170A and the second engagement tip 170B towards each other along the central axis 80. As such, the tool 160 can swage, or plastically deform, the collet lock 140 into the recess 134. An outer surface of the ring 142 or the plurality of fingers 144, or both, can engage the recess 134 in an interference fit, press-fit or snap fit engagement. A tip 148 of the one or more fingers 144 can engage the outer surface of the catheter 90 and compress the catheter 90 either onto the stem 120 or onto an inner surface of the recess 134. Advantageously, the “pinching” action of the tool 160 mitigates applying any pressure directly to the patient, reducing discomfort and reducing the risk of trauma from slipping in the naturally lubricated environment of the subcutaneous placement.

In an embodiment, as shown in FIG. 5, the engagement tip 170 can include an indentation 182. FIG. 5 shows a view of an inner surface 180A of the first arm 162A of the tool 160, with the collet lock 140 aligned with a first indentation 182A. As will be appreciated, the second arm 162A can include a second indentation 182A. A width (w2) of the indentation 182 can be equal to, or larger than, a diameter of the ring 142. As such, the ring 142 can fit within the indentation 182 and can abut laterally or transversely against a side wall thereof. As such, the indentation 182 can ensure a secure fit of the ring 142 with the engagement tip 170 and prevent the tool 160 from slipping when an axial force is applied.

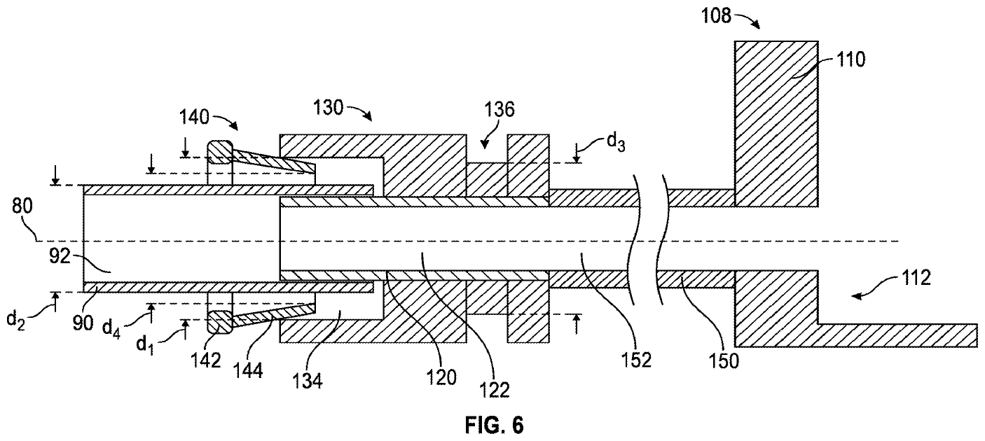

In an embodiment, as shown in FIG. 6, the port system 100 can include an extension leg 150 extending between the port 108 and the cathlock mechanism 130. The extension leg 150 can define an extension leg lumen 152 providing fluid communication between the stem lumen 122 and the reservoir 112. The extension leg can be formed of a rigid material or a flexible material. Advantageously, the extension leg 150 can allow the port 108 to remain stationary while a user can manipulate the catheter 90 and/or the cathlock mechanism 130 to align the catheter 90 with the cathlock 130 and engage therewith, as described herein.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A system for coupling a catheter to a port, comprising:
a catheter defining a first diameter;
a port including a shroud extending axially from the port and defining a recess having a second diameter larger than the first diameter, the recess including a stem extending axially and configured to engage a lumen of the catheter;
a collet lock extending annularly about the stem axis and configured to transition between an unlocked configuration and a locked configuration when an axial force is applied; and
a tool configured to engage the shroud and the collet lock and apply an axial force to transition the collet lock from the unlocked configuration to the locked configuration.

2. The system according to claim 1, wherein a portion of the collet lock is retained within the recess in the unlocked configuration, an outer surface of the collet lock engages an inner surface of the recess in one of an interference fit, press-fit, or snap-fit engagement.

3. The system according to claim 1, wherein the collet lock includes a plurality of fingers configured to plastically deform radially inward when transitioned to the locked configuration.

4. The system according to claim 3, wherein the plurality of fingers impinge on a surface of the recess when the axial force is applied to transition the collet lock from the unlocked configuration to the locked configuration.

5. The system according to claim 3, wherein a tip of a finger of the plurality of fingers engages an outer surface of the catheter in the locked configuration, compressing the catheter onto the stem.

6. The system according to claim 3, wherein a tip of the plurality of fingers define a diameter that is larger than the first diameter of the catheter.

7. The system according to claim 1, wherein the tool includes a first arm hingedly coupled to a second arm, a tip of the first arm includes a first fork configured to engage the port, and a tip of the second arm includes a second fork configured to engage the collet lock.

8. The system according to claim 7, wherein one or both of the first fork and the second fork includes a first tine and a second tine defining a notch having a first width, the first width being equal to or larger than the first diameter of the catheter.

9. The system according to claim 8, wherein the shroud includes a channel defining a third diameter being equal to or less than the first width, the first fork engaging the channel of the shroud in one of an interference fit, press-fit, or snap-fit engagement.

10. The system according to claim 8, wherein one or both of the first fork and the second fork includes an indentation extending along an edge of the notch and defining a second width, the second with being larger than an outer diameter of the collet lock, a surface of the indentation engaging the collet lock with the axial force is applied.

11. The system according to claim 1, further including an extension leg extending between the shroud and the port, formed integrally therewith, and providing fluid communication therebetween, the extension leg formed of a flexible material.

12. The system according to claim 1, wherein the port includes a reservoir in fluid communication with the stem and having a needle penetrable septum thereover.

13-22. (canceled)

23. A catheter and port coupling system, comprising:
a catheter defining a first diameter;
a port including a shroud extending axially from the port and defining a recess having a second diameter larger than the first diameter, the recess including a stem extending axially and configured to engage a lumen of the catheter; and
a collet lock extending annularly about the stem axis and configured to transition between an unlocked configuration and a locked configuration when an axial force is applied to secure the catheter to the port.

24. The system according to claim 23, further including an extension leg extending between the shroud and the port, formed integrally therewith, and providing fluid communication therebetween, the extension leg formed of a flexible material.

25. The system according to claim 23, wherein a portion of the collet lock is retained within the recess in the unlocked configuration, an outer surface of the collet lock engages an inner surface of the recess in one of an interference fit, press-fit, or snap-fit engagement.

26. The system according to claim 23, wherein the collet lock includes a plurality of fingers configured to plastically deform radially inward when transitioned to the locked configuration.

27. The system according to claim 26, wherein the plurality of fingers impinge on a surface of the recess when the axial force is applied to transition the collet lock from the unlocked configuration to the locked configuration.

28. The system according to claim 26, wherein a tip of a finger of the plurality of fingers engages an outer surface of the catheter in the locked configuration, compressing the catheter onto the stem.

29. The system according to claim 26, wherein a tip of the plurality of fingers define a diameter that is larger than the first diameter of the catheter.

30. The system according to claim 23, wherein the port includes a reservoir in fluid communication with the stem and having a needle penetrable septum thereover.

31. The system according to claim 23, further including a tool configured to engage the shroud and the collet lock and apply an axial force to transition the collet lock from the unlocked configuration to the locked configuration.

32. The system according to claim 31, wherein the tool includes a first arm hingedly coupled to a second arm, a tip of the first arm includes a first fork configured to engage the port, and a tip of the second arm includes a second fork configured to engage the collet lock.

33. The system according to claim 32, wherein one or both of the first fork and the second fork includes a first tine and a second tine defining a notch having a first width, the first width being equal to or larger than the first diameter of the catheter.

34. The system according to claim 33, wherein the shroud includes a channel defining a third diameter being equal to or less than the first width, the first fork engaging the channel of the shroud in one of an interference fit, press-fit, or snap-fit engagement.

35. The system according to claim 33, wherein one or both of the first fork and the second fork includes an indentation extending along an edge of the notch and defining a second width, the second with being larger than an outer diameter of the collet lock, a surface of the indentation engaging the collet lock with the axial force is applied.
